(19) United States
(12) Patent Application Publication
(10) Pub. No.: US 2023/0397913 A1
(43) Pub. Date: Dec. 14, 2023

(54) FRICTION FIT ENDOVASCULAR IMPLANT DETACHMENT MECHANISM

(71) Applicant: DePuy Synthes Products, Inc., Raynham, MA (US)

(72) Inventors: Megan GREENWOOD, Raynham, MA (US); Scott SLOSS, Raynham, MA (US); William COHN, Raynham, MA (US)

(73) Assignee: DePuy Synthes Products, Inc., Raynham, MA (US)

(21) Appl. No.: 17/839,630

(22) Filed: Jun. 14, 2022

(51) Int. Cl. A61B 17/12

(52) U.S. Cl. CPC A61B 17/12131; A61B 17/12113; A61B 2017/00477

(57) ABSTRACT

An endovascular detachment mechanism can include an endovascular implant including an open end and a pinched end, a connector positioned approximate the pinched end, a lock wire, and an outer coil surrounding the lock wire. The lock wire can include a distal end engaged within the connector and a threaded portion having a radius larger than the lock wire. The outer coil be engaged to the threaded portion. Axial rotation of the threaded portion can cause the lock wire to translate proximally with respect to the outer coil to thereby disengage the distal end of the lock wire from the connector. The mechanism can include an inner coil and an outer coil, and axial rotation of the inner coil with respect to the outer coil can cause the distal end of the lock wire to disengage from the connector.

### FIELD OF INVENTION

The present invention generally relates to medical instruments, and more particularly, to embolic implants detachment mechanisms for aneurysm therapy.

### BACKGROUND

Cranial aneurysms can be complicated and difficult to treat due to their proximity to critical brain tissues. Recently, tubular braided implants have been introduced that have the potential to treat an aneurysm or other arterio-venous malformation easily, accurately, and safely in a parent vessel without blocking flow into perforator vessels communicating with the parent vessel. Implant devices for treating aneurysms must be delivered through long, small, tortuous blood vessels and positioning must be controlled precisely to ensure aneurysm filling without causing additional occlusions or clotting in nearby vessels. Accordingly, it is necessary to have a delivery and detachment mechanism providing the connection point between a tubular braided implant and a delivery catheter that has the ability to deliver, position, manipulate, and then release the implant.

### SUMMARY

It is an object of the present invention to provide systems, devices, and methods to meet the above-stated needs. Generally, it is an object of the present invention to provide an endovascular detachment mechanism for an endovascular implant. An endovascular treatment system can include the endovascular detachment mechanism and the endovascular implant. The endovascular implant can have an open end and a pinched end. The detachment mechanism can include a connector that is positioned approximate the pinched end. The detachment mechanism can include a lock wire having a distal end engaged within the connector. The lock wire can include a threaded portion that can have a radius larger than the lock wire. The outer coil can surround the lock wire and can be engaged to the threaded portion. Axial rotation of the threaded portion can cause the lock wire to translate proximally with respect to the outer coil. The axial rotation can thereby disengage the distal end of the lock wire from the connector and release the endovascular implant.

In some examples, the connector can be a crimped ferrule.

In some examples, the lock wire distal end can be engaged with the connector with an interference fit.

In some examples, the distal end of the lock wire can have a tapered radius such that translating the lock wire proximally reduces the interference fit between the distal end of the lock wire and the connector.

In some examples, the outer coil can have a first spacing between coils at a proximal portion of the outer coil and a second spacing between coils at a distal portion of the outer coil. The second spacing can be greater than the first spacing.

In some examples, the endovascular treatment system can include a microcatheter that is sized to deliver the endovascular implant to a treatment site while the endovascular implant is in a non-deployed configuration.

In some examples, the lock wire is configured to push the endovascular implant through the microcatheter and to the treatment site.

In some examples, the threaded portion is welded to the lock wire.

In some examples, the endovascular implant can be configured to expand to a deployed configuration to occlude a spherical cavity.

In another aspect, an endovascular treatment system is disclosed. The endovascular treatment system can include an endovascular implant that includes an open end and a pinched end. The detachment mechanism can include a connector positioned approximate the pinched end. The detachment mechanism can include a lock wire having a distal end engaged within the connector. The detachment mechanism can include an inner coil that surrounds the lock wire and is affixed to the lock wire. The detachment mechanism can include an outer coil surrounding the inner coil. Axial rotation of the inner coil with respect to the outer coil can push a distal end of one of the inner coil or the outer coil against approximal end of the connector. The axial rotation can thereby cause the distal end of the lock wire to disengage from the connector to release the endovascular implant.

In some examples, the connector can be a crimped ferrule.

In some examples, the lock wire distal end can be engaged within the connector with an interference fit.

In some examples, the distal end of the lock wire can include a tapered radius such that translating the lock wire proximally can reduce the interference fit between the distal end of the lock wire and the connector.

In some examples, the endovascular treatment system can include a microcatheter that is sized to deliver the endovascular implant to a treatment site while the endovascular implant is in a non-deployed configuration.

In some examples, the lock wire can be configured to push the endovascular implant through the microcatheter and to the treatment site.

In another aspect, a method of constructing an endovascular implant detachment system is disclosed. The method can include providing an endovascular implant that has an open end and a pinched end. The method can include providing a lock wire having a distal end. The method can include welding a threaded portion having a radius larger than the lock wire to the lock wire. The method can include providing a connector positioned approximate the pinched end. The method can include fitting the distal end of the lock wire into the connector. The method can include threading an outer coil over the threaded portion of the lock wire such that axial rotation of the threaded portion is configured to cause the lock wire to translate proximally with respect to the outer coil. The axial rotation can thereby disengage the distal end of the lock wire from the connector and release the endovascular implant.

In some examples, providing the connector can further include crimping a ferrule to the pinched end of the endovascular implant.

In some examples, the connection between the connector and the distal end of the lock wire can be an interference fit.

In some examples, the distal end of the lock wire can include a tapered radius such that translating the lock wire proximally can reduce the interference fit between the distal end of the lock wire and the connector.

In some examples, the method can include providing a microcatheter sized to deliver the endovascular implant to a treatment site while the endovascular implant is in a non-deployed configuration.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an endovascular treatment system including a detachment mechanism including a connector, lock wire with a threaded portion, and an outer coil, according to aspects of the present invention;

FIG. 2A illustrates an endovascular treatment system including an endovascular detachment mechanism securing an implant during delivery through a catheter according to aspects of the present invention;

FIG. 2B is a close-up view of a distal end of a lock wire of the detachment mechanism as indicated in FIG. 2A;

FIG. 2C illustrates a detachment sequence of the implant by manipulating the detachment mechanism, according to aspects of the present invention;

FIGS. 3A and 3B illustrate a detachment sequence of an example implant by manipulating a detachment mechanism, according to aspects of the present invention;

FIGS. 4A and 4B illustrate a detachment sequence of an example implant by manipulating detachment mechanism, according to aspects of the present invention; and

FIG. 5 is a flowchart of a method for constructing an endovascular treatment system, according to aspects of the present invention.

### DETAILED DESCRIPTION

Examples presented herein generally include a detachment mechanism that can be used with a braided implant that can be secured within an aneurysm sac and occlude a majority of the aneurysm's neck. The implant can include a tubular braid that can be set into a predetermined shape, compressed for delivery through a microcatheter, and implanted in at least one implanted position that is based on the predetermined shape and the geometry of the aneurysm in which the braid is implanted. When compressed, the implant can be sufficiently short to mitigate friction forces produced when the implant is delivered unsheathed through the microcatheter allowing for a more simplistic delivery system compared to some other known braided embolic implant delivery systems. The implant can be as described in U.S. Pat. No. 10,653,425, the entirety of which is incorporated herein by reference as if included in full.

The endovascular implant can include memory shape material that can be heat set to a predetermined shape, can be deformed for delivery through a catheter, and can self-expand to an implanted shape that is based on the predetermined shape and confined by the anatomy of the aneurysm in which it is implanted.

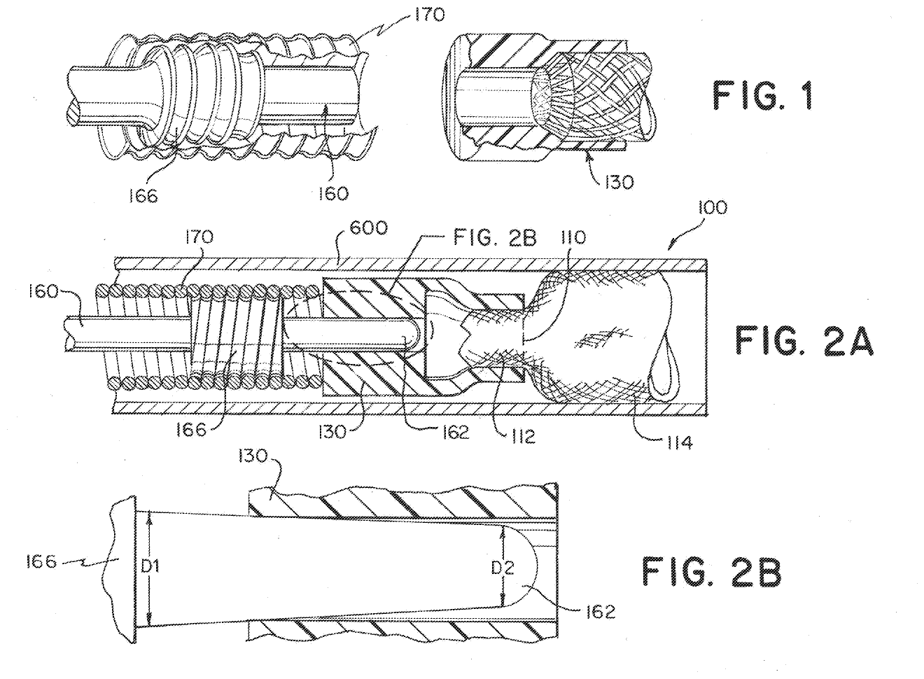

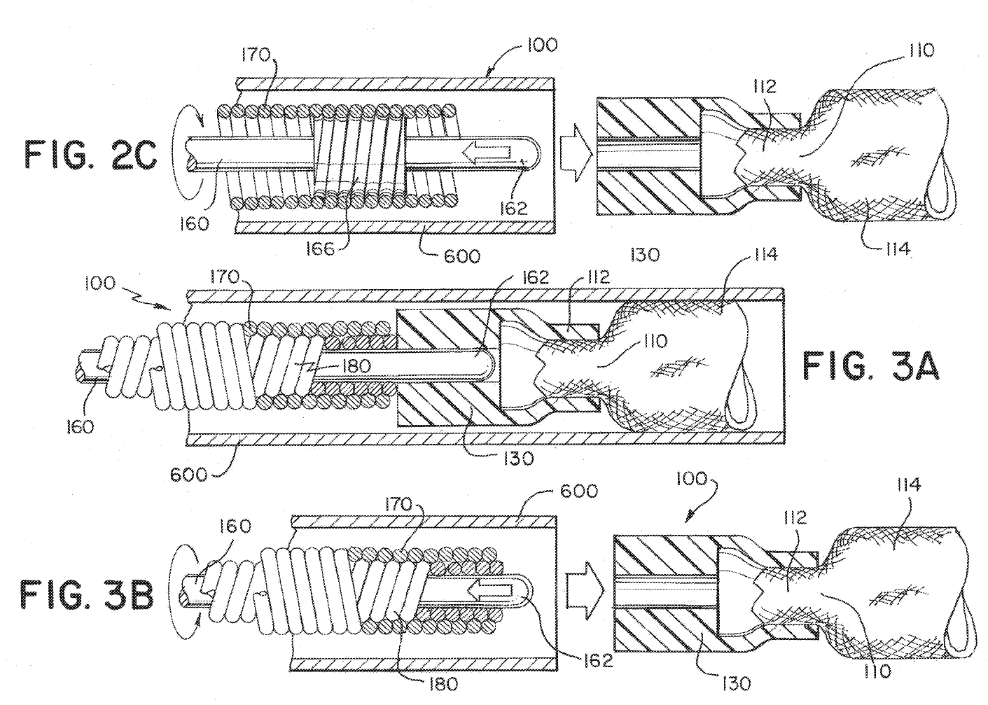

FIGS. 1 and 2A illustrate a detachment mechanism including a connector, lock wire with a threaded portion, and an outer coil. As shown, an example detachment mechanism can include a lock wire 160 that has a distal end 162 and a threaded portion 166, an outer coil 170 engaged to the threaded portion 166, and a connector 130. The distal end of the connector 130 can be configured to receive a pinched end 112 of an endovascular implant 110. The connector 130 can be a metal ferrule that can be crimped to fasten the pinched end 112 to the connector 130. The lock wire 160 distal end 162 can fit into a proximal end of the connector 130. The connector can then be crimped to fasten the lock wire 160 distal end 162 to the proximal end of the connector 130, thereby forming an interference fit in which the distal end 162 is held inside connector 130 due to frictional force. The outer coil 170 can be rotated axially with respect to the lock wire 160. The axial rotation of the outer coil 170 with respect to the lock wire 160 can cause the lock wire to be translated proximally with respect to the connector 130 and the outer coil 170, thereby allowing the distal end 162 to overcome the interference fit with the connector 130 and exit the connector 130.

FIG. 2A shows a endovascular treatment system 100 including the lock wire 160, connector 130, and implant 110 being delivered through microcatheter 600 to a treatment site. In some examples, the lock wire 160 distal end 162 can be tapered.

As illustrated in FIG. 2B, at a distal portion of distal end 162, the diameter of the lock wire 160 can be D2, whereas at a more proximal point of the distal end 162 of the lock wire 160, the diameter of the lock wire 160 can be D1, which is greater than diameter D2. By having a tapered distal end 162, the lock wire 160 can be more easily disconnected from the connector 130 by reducing the frictional force between the lock wire 160 distal end 162 and the connector 130 as the lock wire 160 is translated proximally with respect to connector 130.

FIG. 2C illustrates a detachment sequence of an example implant by manipulating detachment mechanism. As shown, lock wire 160 can be rotated with respect to the outer coil 170. As lock wire 160 is rotated with respect to the outer coil 170, the threaded portion 166 interfaces with the outer coil 170 and can cause the lock wire 160 to be translated proximally with respect to the connector 130. In a different embodiment, the lock wire 160 can have a non-tapered diameter, while the connector can have a tapered internal diameter such that the connector 130 decreases in internal diameter in a distal direction. When the implant 110 is disengaged, the connector 130 remains attached to the pinched end 112 of the braid, and thereby remains implanted in a patient during treatment. The catheter 600, lock wire 160, and outer coil 170 can be extracted from the patient.

FIGS. 3A and 3B illustrate a detachment sequence of an example implant by manipulating a detachment mechanism. FIG. 3A shows a detachment mechanism including the lock wire 160, connector 130, and implant 110 being delivered through microcatheter 600 to a treatment site. The detachment sequence of FIGS. 3A and 3B can be similar to the sequence described with respect to FIGS. 2A and 2B, except that the detachment mechanism can include an outer coil 170 and an inner coil 180 in place of threaded portion 166. The inner coil 180 can be attached to the lock wire 160 by any known means, for example, via welds, glues, and/or the like such that rotating lock wire 160 causes inner coil 180 to rotate with respect to outer coil 170. As inner coil 180 rotates with respect to outer coil 170, the lock wire 160 can be translated proximally with respect to connector 130.

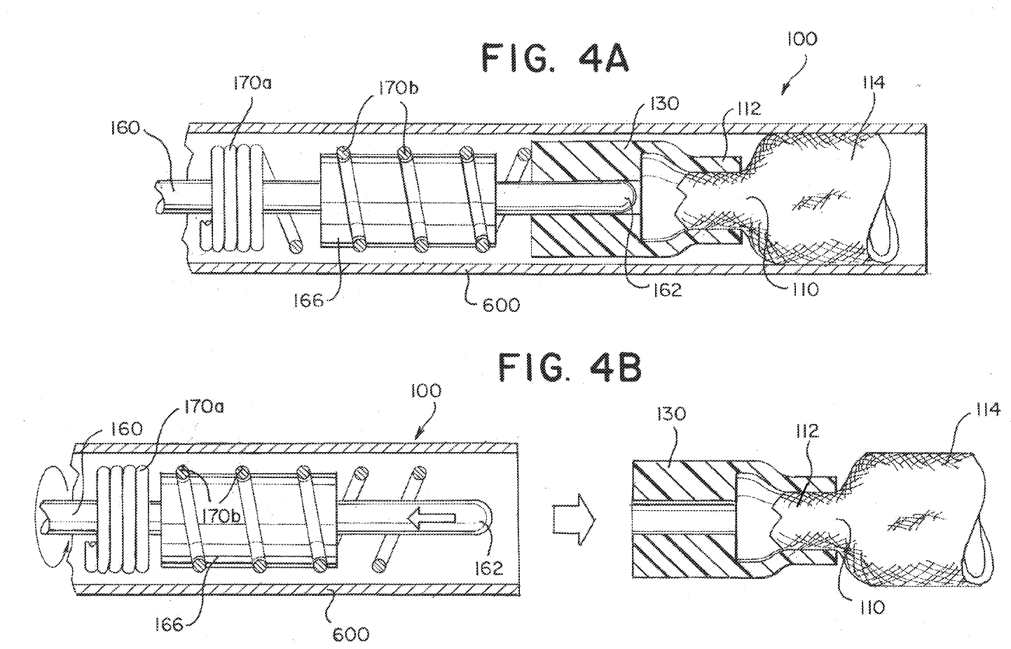

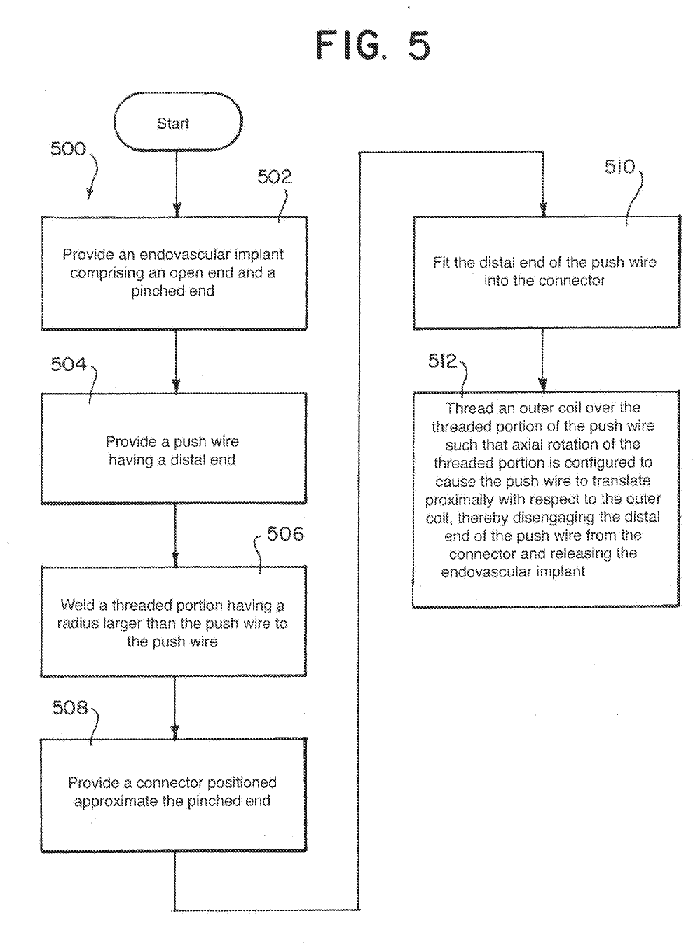

FIGS. 4A and 4B illustrate a detachment sequence of an example implant by manipulating a detachment mechanism. FIG. 4A shows an endovascular treatment system 100 including the lock wire 160, connector 130, and implant 110 being delivered through microcatheter 600 to a treatment site. The detachment sequence of FIGS. 4A and 4B can be similar to the sequence described with respect to FIGS. 2A and 2B, except that the detachment mechanism can include an outer coil 170 with a varied coil spacing. The outer coil can have an open spacing 170b approximate a distal end 162 of lock wire, and a tight spacing 170a at a proximal end of outer coil 170. Accordingly, the threaded portion 166 can be configured to interface with the section of outer coil 170b having the greater coil spacing. As compared to the detachments system of FIGS. 2A and 2B, the detachment system of FIGS. 4A and 4B can allow an operator of the detachment system to make fewer rotations of the lock wire 160 before the lock wire 160 detaches from connector 130. As lock wire 160 rotates with respect to outer coil 170 (e.g., open spacing 170b), the lock wire 160 can be translated proximally with respect to connector 130.

FIG. 5 is a flowchart of a method for constructing an endovascular treatment system. In block 502, the method can include providing an endovascular implant 110. The implant can be a braided tubular implant that includes an open end 114 and a pinched end 112. The open end may be distal to the pinched end.

In block 504, the method can include providing a lock wire 160 having a distal end 162.

In block 506, the method can include welding a threaded portion 166 to the lock wire. The threaded portion can have a radius larger than the lock wire 160.

In block 508, the method can include providing a connector 130 positioned approximate the pinched end 112. In some examples, the connector can be a ferrule that can be crimped to the lock wire. The ferrule can be constructed of any suitable material, such as a metal alloy.

In block 510, the method can include fitting the distal end 162 of the lock wire 160 into the connector 130. That is the distal end 162 of lock wire can be pushed into connector 130, and the connector 130 can be tightly crimped over the distal end of 162, thereby forming an interference fit.

In block 512, the method can include threading an outer coil 170 over the threaded portion 166 of the lock wire. The outer coil 170 can be rotated axially with respect to the threaded portion 166 such that the lock wire is translated proximally with respect to the outer coil 170. In response to the axial rotation, the distal end 162 of the lock wire 160 can disengage from the connector and release the endovascular implant 110.

The tubular braid of the example implant 110 can include memory shape material that can be heat set to a predetermined shape, can be deformed for delivery through a catheter, and can self-expand to an implanted shape that is based on the predetermined shape and confined by the anatomy of the aneurysm in which it is implanted.

The example implant described herein can rely on a radial outward force to anchor the implant within the sac of an aneurysm. To this end, the braid 110 can be shaped to a predetermined shape having a diameter that is greater than its height so that the braid is radially constricted when implanted in an aneurysm. The ratio of diameter to height of the braid 110 in a respective predetermined shape can be within the range of 2:1 to 1:3 to treat aneurysms of many known sizes and shapes.

The descriptions contained herein are examples of embodiments of the invention and are not intended in any way to limit the scope of the invention. As described herein, the invention contemplates many variations and modifications of the implant, including alternative materials, alternative geometries, alternative detachment features, alternative delivery systems, alternative means for forming a braid into a predetermined shape, alternative treatment methods, etc. These modifications would be apparent to those having ordinary skill in the art to which this invention relates and are intended to be within the scope of the claims which follow.

What is claimed is:

1. An endovascular treatment system, comprising:
an endovascular implant comprising an open end and a pinched end;
a connector positioned approximate the pinched end;
a lock wire having a distal end engaged within the connector, the lock wire comprising a threaded portion having a radius larger than the lock wire; and
an outer coil surrounding the lock wire and engaged to the threaded portion,
wherein axial rotation of the threaded portion causes the lock wire to translate proximally with respect to the outer coil, thereby disengaging the distal end of the lock wire from the connector and releasing the endovascular implant.

2. The endovascular treatment system of claim 1, wherein the connector comprises a crimped ferrule.

3. The endovascular treatment system of claim 1, wherein the distal end of the lock wire is engaged within the connector with an interference fit.

4. The endovascular treatment system of claim 3, wherein the distal end of the lock wire comprises a tapered radius such that translating the lock wire proximally reduces the interference fit between the distal end of the lock wire and the connector.

5. The endovascular treatment system of claim 1, wherein the outer coil comprises a first spacing between coils at a proximal portion of the outer coil and a second spacing between coils at a distal portion of the outer coil, the second spacing greater than the first spacing.

6. The endovascular treatment system of claim 1, further comprising a microcatheter sized to deliver the endovascular implant to a treatment site while the endovascular implant is in a non-deployed configuration.

7. The endovascular treatment system of claim 6, wherein the lock wire is configured to push the endovascular implant through the microcatheter and to the treatment site.

8. The endovascular treatment system of claim 1, wherein the threaded portion is welded to the lock wire.

9. The endovascular treatment system of claim 1, wherein the endovascular implant is configured to expand to a deployed configuration to occlude a spherical cavity.

10. An endovascular treatment system, comprising:
an endovascular implant comprising an open end and a pinched end;
a connector positioned approximate the pinched end;
a lock wire having a distal end engaged within the connector;
an inner coil surrounding the lock wire and affixed to the lock wire; and
an outer coil surrounding the inner coil,
wherein, axial rotation of the inner coil with respect to the outer coil pushes a distal end of one of the inner coil or the outer coil against a proximal end of the connector, thereby causing the distal end of the lock wire to disengage from the connector to release the endovascular implant.

11. The endovascular treatment system of claim 10, wherein the connector comprises a crimped ferrule.

12. The endovascular treatment system of claim 10, wherein the distal end of the lock wire is engaged within the connector with an interference fit.

13. The endovascular treatment system of claim 12, wherein the distal end of the lock wire comprises a tapered radius such that translating the lock wire proximally reduces the interference fit between the distal end of the lock wire and the connector.

14. The endovascular treatment system of claim 10, further comprising a microcatheter sized to deliver the endovascular implant to a treatment site while the endovascular implant is in a non-deployed configuration.

15. The endovascular treatment system of claim 14, wherein the lock wire is configured to push the endovascular implant through the microcatheter and to the treatment site.

16. A method of constructing an endovascular treatment system, comprising:
providing an endovascular implant comprising an open end and a pinched end;
providing a lock wire having a distal end;
welding a threaded portion having a radius larger than the lock wire to the lock wire;
providing a connector positioned approximate the pinched end;
fitting the distal end of the lock wire into the connector; and
threading an outer coil over the threaded portion of the lock wire such that axial rotation of the threaded portion is configured to cause the lock wire to translate proximally with respect to the outer coil, thereby disengaging the distal end of the lock wire from the connector and releasing the endovascular implant.

17. The method of claim 16, wherein providing the connector further comprises crimping a ferrule to the pinched end of the endovascular implant.

18. The method of claim 16, wherein fitting the distal end of the lock wire into the connector further comprises an interference fit.

19. The method of claim 18, wherein the distal end of the lock wire comprises a tapered radius such that translating the lock wire proximally reduces the interference fit between the distal end of the lock wire and the connector.

20. The method of claim 16, further comprising providing a microcatheter sized to deliver the endovascular implant to a treatment site while the endovascular implant is in a non-deployed configuration.
